(19) United States
(12) Patent Application Publication
(10) Pub. No.: US 2005/0010185 A1
(43) Pub. Date: Jan. 13, 2005

(54) Auxiliary sheet for absorbable article

(75) Inventors: Satoshi Mizutani, Kagawa (JP); Koichi Yamaki, Kagawa (JP); Yuki Noda, Kagawa (JP)

Correspondence Address: DARBY & DARBY P.C., P. O. BOX 5257, NEW YORK, NY, 10150-5257, US

(73) Assignee: Uni-Charm Corporation, Kawanoe-Shi (JP)

(21) Appl. No.: 10/888,539

(22) Filed: Jul. 8, 2004

Related U.S. Application Data
(63) Continuation of application No. PCT/JP02/12015, filed on Nov. 18, 2002.

(51) Int. Cl. A61F 13/15; A61F 13/20

(52) U.S. Cl. 604/385.03; 604/385.101

(57) ABSTRACT

The present invention relates to an auxiliary sheet for an absorbent article which supplements function of a sheet-form absorbent article such as a sanitary napkin, or which adds new function to the sheet-form absorbent article, and provides an auxiliary sheet for an absorbent article which reduces a contact range between a body fluid absorbed in the sheet-form absorbent article and the body of a wearer and also prevents the body fluid from diffusing, and further prevents the skin from being re-wetted and also prevents the body fluid from being caused to leak out of the absorbent article.

The auxiliary sheet for the absorbent article (1) provided with a through-hole (2) permitting to engage labia therein and an adhesive portion (3) for fixedly adhering to the body of the wearer in the neighborhood of the circumferential edge of this through-hole (2), is made to lie between a sanitary napkin (10) and the thighs of the wearer.

### CROSS-REFERENCE TO RELATED APPLICATIONS

This application is a Continuation of International Application No. PCT/JP2002/012015 filed Nov. 18, 2002, which application published in Japanese on Jun. 3, 2004 as WO 2004/045476 A1 under PCT Article 21 (2).

### FIELD OF THE INVENTION

The present invention relates to an auxiliary sheet for an absorbent article which supplements a sheet-form absorbent article such as a sanitary napkin, or provides the sheet-form absorbent article with a new function.

### BACKGROUND ART

Conventionally, a sanitary napkin has widely been used as a sheet-form absorbent article for absorbing a body fluid such as menstrual blood. A great effort has been made for such a sanitary napkin to prevent the menstrual blood from leaking. Moreover, the sanitary napkin has been provided with various contrivances for improving a wearing feeling of the sanitary napkin which absorbed menstrual blood while it is worn.

Concerning this point, as a sanitary napkin improved in prevention of leak and a feeling of wear, the Japanese Utility Model Laid-Open No. 1995-18712 proposed a sanitary napkin that was provided with a protruding portion behind to fix it in the labia, and was also provided with a gap in front across the clitoris so as to be separated (refer to FIG. 7 in the same Publication). The use of this sanitary napkin makes it possible to directly absorb menstrual blood excreted from the ostium vaginae into the protruding portion, therefore, the use of the sanitary napkin can reduce adhesion of leaking menstrual blood to underwear or the like. Moreover, since the sanitary napkin is not in contact with the vicinity of clitoris, the sanitary napkin does not rub the vicinity of the clitoris even when the sanitary napkin attached to the underwear is moved, and inflammation by rubbing is prevented from occurring.

Here, as to a sanitary napkin, in general, the surface is saturated with menstrual blood when it has absorbed a certain quantity of menstrual blood, and the menstrual blood will no longer be absorbed further inside. Therefore, when a menstrual blood absorption capacity has been saturated in the above protruding portion in the sanitary napkin related to the Japanese Utility Model Laid-Open No. 1995-18712, said protruding portion becomes hard to absorb the menstrual blood, and the menstrual blood not absorbed therein flows out forwardly along the pudendal slit, and is in danger of leaking out of the gap provided between the sanitary napkin and the clitoris.

Moreover, since the above protruding portion is to be fixed in labia, the menstrual blood contained in said protruding portion always brings the labia into wetting state. Moreover, the labia have an internal membrane formed of a mucous membrane, therefore, they have little resistance to stimulus and will easily have rough skin and a rash due to long time contact with the menstrual blood compared with other portions.

Furthermore, the menstrual blood once absorbed in the sanitary napkin turns back into the body side due to a wearing pressure or the like, and this may cause, what is called, re-wetting. In such a case, in the portion where the wearer's skin is in close contact with the sanitary napkin, to be more specific, in the range where the menstrual blood is absorbed by the above protruding portion, rough skin and a rash are in danger of being caused.

As a sanitary napkin for preventing such re-wetting, the JP-A-1999-507577 discloses a sanitary napkin with a double layer top sheet including a first layer provided with a perforated film having an opening larger than that to be arranged in a sheet of an ordinary sanitary napkin, and a second layer provided with a perforated film without the large opening. Owing to the large opening, the use of this sanitary napkin allows the first layer having a high liquid transmission rate to speedily transmit menstrual blood and to transmit the blood to the second layer. Then, the second layer having a liquid transmission rate lower than that of the first layer shields the absorbed menstrual blood, to prevent it from flowing backward, therefore, the occurrence of re-wetting seems to be reduced. However, the menstrual blood absorbed in the first layer may sometimes turns back to the first layer without being transmitted to the second layer, and this causes re-wetting in such a case.

The present invention has been made considering the subject described above, and the object of the invention is to provide an auxiliary sheet for an absorbent article which permits not only to reduce a contact range in which the menstrual blood absorbed on a sheet-form absorbent article comes into contact with the body of a wearer, but also to prevent the menstrual blood from diffusing, and further to reduce re-wetting of the skin and the occurrence of leakage of the menstrual blood from the absorbent article.

### DISCLOSURE OF THE INVENTION

In order to solve the subjects as the above, the present invention is characterized in that the diffused menstrual blood is prevented from coming into direct contact with the body of a wearer and an occurrence of a rash or the like can be avoided, by providing such an auxiliary sheet for an absorbent article as is to be laid between a sheet-form absorbent article and the inside of the thighs of a wearer and has a structure provided with a through-hole permitting to engage the labia therein so that the menstrual blood leaking from the inside of the labia can speedily be guided from the through-hole into the absorbent article when the labia are engaged in the through-hole.

To be more specific, the present invention provides an auxiliary sheet for an absorbent article as the following.

(1) An auxiliary sheet for an absorbent article which is to be used together with a sheet-form absorbent article, and supplements the functions of a sheet-form absorbent article or provides new functions to the sheet-form absorbent article, said auxiliary sheet for the absorbent article is provided with a through-hole for smoothly engaging therein a predetermined range of a pair of labia, and a depth of said through-hole is set deeper than that of a pair of said labia to be engaged in said through-hole when the through-hole of said auxiliary sheet for the absorbent article is engaged with a pair of said labia.

The auxiliary sheet for an absorbent article in accordance with the present invention is provided with a through-hole of such a size that can engage labia therein, therefore, a body fluid (especially menstrual blood) leaking from the inside of labia is speedily guided into the sheet-form absorbent article by putting the labia into the through-hole when wearing it. Thus, the body fluid is not diffused in the state in which the body fluid is in contact with the body between the wearer's body and the auxiliary sheet for the absorbent article.

Moreover, since the auxiliary sheet for the absorbent article in accordance with the present invention is the one to be laid between the sheet-form absorbent article and the body of a wearer, clearance is created between the sheet-form absorbent article and the body of a wearer within such an interjacent range. Therefore, even if the body fluid once absorbed into the sheet-form absorbent article flows back to the body side face due to the wear pressure, it is possible to remarkably reduce the occurrence of re-wetting the skin of the wearer.

Further, according to the auxiliary sheet for the absorbent article in accordance with the present invention, the above through-hole depth has such a dimension as the tips of the labia are made hard to come into contact with the sheet-form absorbent article placed on the auxiliary sheet for the absorbent article on the opposite side of the body even when the labia are engaged in such a through-hole. Therefore, such a situation as the labia are in contact with the body fluid for a long time as in a conventional example, is hard to occur.

Here, since Japanese women have about a 17 mm depth dimension of labia as an average value, it is recommended that a depth of the through-hole is made in the range of 3 to 30 mm, preferably, in the range of 5 to 25 mm, more preferably, in the range of 10 to 20 mm. The non-contact state between the sheet-form absorbent article and the labia of a wearer is surely secured by arranging the through-hole in such a dimensional range of the depth.

Moreover, in the auxiliary sheet for the absorbent article in accordance with the present invention, it is applicable as long as the depth of the through-hole is deeper than that of labia, and the whole auxiliary sheet for the absorbent article does not need to be as thick as the depth of the through-hole. Therefore, it is enough for the periphery of the through-hole to have substantially the same thickness as the depth of the through-hole, and the other portion may be thinner than that.

Thus, when the auxiliary sheet for the absorbent article in accordance with the present invention is used, the flow of the body fluid is oriented so as to pass through the through-hole provided in the auxiliary sheet for the absorbent article. Therefore, a rough dry skin and a rash due to the contact of the wearer's body with the sheet face full of the body fluid can be suppressed.

Moreover, since the body fluid is absorbed into the sheet-form absorbent article as it is from the inside of labia via the through-hole, such a situation is remarkably reduced as the body fluid is diffused in a wide range and leaks outside of the sheet-form absorbent article. Therefore, this is able to give psychogenic calmness to the wearer.

As described above, the auxiliary sheet for the absorbent article in accordance with the present invention is capable of securing the function of preventing a rough dry skin and leakage of the body fluid for the sheet-form absorbent article.

In addition to the above, even if the sheet-form absorbent article to be used together with the auxiliary sheet for the absorbent article should lose shape, the flow of the body fluid is oriented only in a predetermined direction due to the existence of the through-hole, therefore, the danger of the body fluid leakage is reduced.

(2) The auxiliary sheet for the absorbent article according to (1), wherein said auxiliary sheet for the absorbent article comprises at least one layer, said layer comprises at least one of a liquid impermeable sheet and a soft touch sheet.

The auxiliary sheet for the absorbent article like this permits to involve a liquid impermeable sheet. Therefore, even when the body fluid (especially, menstrual blood) once absorbed into the sheet-form absorbent article flows back to the auxiliary sheet body for the absorbent article, the body fluid is obstructed by the liquid impermeable sheet and is prevented from infiltrating into the body side face of the auxiliary sheet for the absorbent article. Namely, even if the body fluid once absorbed into the sheet-form absorbent article performs re-wetting due to wearing pressure or the like, the re-wetting is not caused to the wearer's skin within the range in which the auxiliary sheet for the absorbent article in accordance with the present invention is laid in-between.

As a material for the “liquid impermeable sheet”, a liquid impermeable film or a water-resistant sheet, or a laminated material of the sheet and nonwoven fabric is mostly used. To be more concrete, it is a film of 20 to 30 g/m² molded from PP (polypropylene), PE (polyethylene), PET (polyethylene terephthalate), or the like. Moreover, when a feeling is considered, it is preferred to use a film primarily comprising a low density polyethylene (LDPE) resin.

Other liquid impermeable sheets include a single sheet made of PE, PP, or a random copolymer resin of PP and PE, of a spun bonded nonwoven fabric (S) made of a bicomponent fiber forming a sheath-core structure such as PE/PP and PE/PET, of a melt-blown nonwoven fabric (M), or a composite nonwoven fabric sheet such as SMS and SSMMSS combining S and M, or the like. To be more specific, the sheet is a combination of the layer S of 5 to 15 g/m² and the layer M of 15 to 30 g/m² in the rate, and a SMS sheet having a water resistant pressure of 3000 Pa or higher is preferred for the use.

Further, as another liquid impermeable material, a foam material can be cited. For the foam material, a material having an independent foam structure is used. There is a preferable form material of 1.5 mm in thickness obtained by foaming a resin of PE/EVA mixed in the ratio of 80 to 20 at a magnification of 15 times and then cross-linking them by irradiating it with electrons, cutting it in half in the direction of thickness, making the front side as a skin layer and the inner side as a cell layer, and coating the inner side with a surface active agent.

In the auxiliary sheet for the absorbent article in accordance with the present invention, a soft touch sheet may be placed on the side of the skin-contact surface. In such an arrangement, a comfortable feeling can be obtained when the skin is in contact with the auxiliary sheet for the absorbent article, and also a rough dry skin and a rash can be reduced.

Moreover, in the present specification, “soft touch” means a soft and comfortable feeling, and a high and cushiony sheet, a smooth sheet giving no friction to the skin, a sheet having expansion and contraction or tensibility capable of following up skin movement, or the like can be properly selected for the use.

The liquid impermeable sheet and the soft touch sheet can singly be provided on the auxiliary sheet for the absorbent article in accordance with the present invention, however, both can be provided thereon for forming a multi-layer structure.

As a multi-layer structure, for example, each layer of the liquid impermeable sheet and the soft touch sheet is placed so as to be of a two-layer structure, and a three-layer structure is also possible by placing the soft touch sheets on the skin contact surface and the contact surface of the sheet-form absorbent article, respectively, and sandwiching the impermeable sheet in-between.

In the case of the three-layer structure, the soft touch sheet placed on the body side face firstly comes into soft contact with the skin of a wearer when wearing, therefore, a comfortable wearing feeling is provided. On the other hand, the soft touch sheet placed on the opposite body side face to body side face absorbs the flow of the body fluid re-wetted by wear pressure when wearing the sheet, to reduce the occurrence of leakage of the body fluid.

(3) The auxiliary sheet for the absorbent article according to (2), wherein the liquid impermeable sheet has air permeability.

A material having air permeability is used for the liquid impermeable sheet provided on the auxiliary sheet for the absorbent article in accordance with the present invention. Therefore, even a long time wearing does not cause to get steamy.

What is called air permeable film is available for a material of the liquid impermeable sheet having “air permeability”, where innumerable cells are formed by mixing an inorganic filler at the rate of 40 through 60 weight % and providing elongational processing. It is preferred to use this film in the range of 20 to 40 g/m².

For an air permeable material, a three-dimensional foam film can also be used. For this three-dimensional film, a film of such a structure as has air permeability and is also able to prevent a liquid from infiltrating is used, especially, a film of such a shape as the height is 0.3 to 1.0 mm; an opening rate is 20 to 50%; the hole diameter is 0.3 to 0.6 mm; and a capillary tube is tapered or tilted, is selected. The selected three-dimensional form film is to be placed so that the capillary tubes are faced to underwear. Moreover, water repellant agent such as silicone, fluorine, etc. can be mixed in the three-dimensional form film at the ratio of 0.1 through 5%.

(4) The auxiliary sheet for the absorbent article according to (2) or (3), wherein said soft touch sheet comprises at least one of a nonwoven fabric sheet and a foam sheet.

According to the auxiliary sheet for the absorbent article in accordance with the present invention, the soft touch sheet is composed at least one of a nonwoven fabric sheet and a foam sheet.

The auxiliary sheet for the absorbent article provided with such a soft touch sheet on the body side gives a smooth feeling to a wearer, and reduces a rough dry skin, a rash, or the like further, and can also improve the wearing feeling.

As a material for a “nonwoven” sheet, a single or a laminated fiber sheet or the like can be used. As the fiber sheet, for example, such a 1.1 to 3.3 dtex fiber can be cited as has a sheath-core structure such as PE/PP, PE/PET, or the like provided with water repellant processing on the surface with silicone, fluorine compound, or the like is formed into a fiber web by means of the card method, and is sheeted by a heat embossing process or through-air or water-flow interlacing.

For the nonwoven fabric sheet, a mixture with reproduced/natural cellulose fiber of 5 to 30 weight % can be used. This mixture makes it possible to reduce a steamy feeling from the wearer's skin.

As other materials for the nonwoven fabric sheet, as a single sheet, PP and PE, a random copolymer resin of PP and PE, spun bonded nonwoven fabric (S) made of bicomponent fiber forming a sheath-core structure like PE/PP and PE/PET, melt-blown nonwoven fabric (M), SMS, SSMMSS, which are composite materials of S and M, or the like are can be used.

As a preferable example of a nonwoven fabric, through-air nonwoven fabric can be cited, which feels especially smooth and is excellent in a bulky feeling. To be more concrete, the through-air nonwoven fabric is composed of 90 weight % fiber made to 2.2 dtex by curling the bicomponent fiber of PE/PP crimpy and 10 weight % of natural cellulose fiber, by a specific weight per unit of 30 g/m² and 1.2 mm of height. Other than the above, a lamination of a film primarily comprising synthetic rubber and nonwoven fabric can also be used.

Further as other materials, a three-dimensional foam film, a lamination of a film primarily comprising PE or synthetic rubber and nonwoven fabric, and a foam sheet primarily comprising PE or synthetic rubber can be used. For example, a closed cell structure, an open cell structure, or the like can be cited. As a foam sheet, especially, a foam sheet is preferable, having a flexible closed cell structure, being bridged by electron radiation after having foamed a mixture primarily comprising PE or a mixture of PE and EVA, being 0.5 to 2.0 mm in thickness, and having a foaming magnification of 5 to 20 times. Moreover, in the case of using the three-dimensional foam film, it is preferable to place the film with the capillary existent surface faced to the side of the skin abutment surface.

(5) The auxiliary sheet for the absorbent article according to any one of (1) to (4), wherein said through-hole has an oblong elliptic opening.

According to the auxiliary sheet for the absorbent article in accordance with the present invention, the opening of the through-hole is formed long elliptical. Therefore, a wearer is easily able to put the longitudinal shape labia into the through-hole, and can also orient the flow of the body fluid (especially, menstrual blood) traveling along the labia to the sheet-form absorbent article. As a result, the diffusion of the body fluid flowing from the inside of the labia can be prevented better.

As to the opening of the through-hole, a longitudinal dimension is preferred to be within the range of 60 to 180 mm, more preferably, within the range of 80 to 150 mm, furthermore preferably, within the range of 90 to 120 mm. Moreover, the dimension of the width is preferred to be within the range of 30 to 80 mm, more preferably, within the range of 35 to 50 mm. It becomes possible to suitably engage a pair of labia in the through-hole by forming the opening of the through-hole within such ranges as the above.

(6) The auxiliary sheet for the absorbent article according to any one of (1) to (5), wherein said through-hole is biased toward either side of both end portions in the longitudinal direction of said auxiliary sheet for the absorbent article.

According to the auxiliary sheet for the absorbent article in accordance with the present invention, the through-hole is arranged in a portion off the central portion in the longitudinal direction. This is an arrangement considering the presence of pubic hairs.

Namely, in the area (pubic hair portion) where many pubic hairs are present, the pubic hairs obstruct close contact between the auxiliary sheet for the absorbent article and the body of a wearer, therefore, a gap is prone to generate between the auxiliary sheet for the absorbent article and the body of the wearer.

Moreover, since pubic hairs are much thicker than the fiber used for the nonwoven fabric and have large surface areas, the body fluid is prone to adhere to the pubic hairs compared with the fiber used for the nonwoven fabric. Moreover, since the pubic hairs have minute ruggedness (cuticles), the body fluid once adhering thereto is caught in the ruggedness and becomes difficult to be separated from the pubic hairs. This results in diffusion of the body fluid traveling along the pubic hairs, and the body fluid is in danger of leaking out of the gap generating between the auxiliary sheet for the absorbent article and the body of the wearer as described above.

As to this matter, it may be considered that the above-described gap can be prevented from generating and the body fluid can be prevented from leaking by closely adhering the auxiliary sheet for the absorbent article to the body of a wearer with an adhesive. However, when the adhesive is directly applied to the pubic hair portion, the pubic hairs are pulled together with the auxiliary sheet for the absorbent article at the time of removing it, and the skin is in danger of being hurt. When the adhesive is applied evading the pubic hairs to avoid the above matter, the production of a gap in the pubic hair portion cannot be prevented, and this method is insufficient to prevent the body fluid from leaking.

Concerning this point, the auxiliary sheet for the absorbent article in accordance with the present invention is provided with the through-hole not in the center in the longitudinal direction but on one or the other side of both end portions, therefore, a contact range between the pubic hair portion and the plane portion of the auxiliary sheet for the absorbent article is widen. As a result of this, the body fluid flowing via the pubic hairs is shifted to the side of the auxiliary sheet for the absorbent article, and this permits to prevent the leakage of the body fluid accompanied by the diffusion of the body fluid, therefore, the danger of a rough dry skin can be reduced. Moreover, since the pubic hairs are generally present more in front of labia, it is preferable that the through-hole is positioned near the rear end portion of the auxiliary sheet for the absorbent article (refer to FIG. 4).

To take the body fluid diffusing in the pubic hair portion, it is effective not only to expand the contact range of the auxiliary sheet for the absorbent article and the pubic hair portion in the longitudinal direction by elongating the through-hole, but also to shape the auxiliary sheet for the absorbent article like a rice scoop by widening the area near the front end portion to expand the range also in the direction of the width (refer to FIG. 5). By shaping the auxiliary sheet for the absorbent article like this, it is possible to further reduce anxiety of a wearer about leakage of the body fluid.

Moreover, in the present specification, the “front end portion” of the auxiliary sheet for the absorbent article means the portion positioned near the mons veneris when wearing it, and the “rear end portion” means the portion positioned near the perineum.

(7) The auxiliary sheet for the absorbent article according to any one of (1) to (6), having an adhesive portion for adhering to body of a wearer on a body side face of the auxiliary sheet for the absorbent.

According to the auxiliary sheet for the absorbent article as the above in accordance with the present invention, the adhesive portion is arranged on the side face in contact with the body. Therefore, it is possible to adhere the auxiliary sheet for the absorbent article to the body by bringing the adhesive applied to such an adhesive portion into contact with the body. Thus, the auxiliary sheet for the absorbent article is prevented from being dislocated as a wearer moves, therefore, it is possible to keep the suitable worn state when it was put on.

The adhesive portion can take various shapes like a belt, an ellipse, a half ellipse, or the like, and also a plurality of adhesive portions can be provided.

And, the allocation of an adhesive on the adhesive portion is not specially restricted, but it can properly be selected to be striped, flat, dotted, mesh-like, or the like.

As to the adhesion of an adhesive, the measured values are preferred to have a peel strength in the range of 100 to 2000 mN and a shear force in the range of 3000 to 15000 mN when an adhesive on a polyester film is pressed to stick on a stainless plate weighted by a roller of a 3000 Pa load thereon, and thereafter a 180 degree peel strength is measured at speed of testing rate of stressing of 100 m/min. These values are the ones considering a burden on the skin.

Moreover, to prevent the adhesive from sticking to each other, it is recommended that the feeling of the auxiliary sheet for the absorbent article has a measured value in the range of 10 to 70 mm/25 mm, more preferably, in the range of 15 to 40 mm/25 mm according to JIS L-1018 (Cantilever Method A). By providing the auxiliary sheet for the absorbent article with such a feeling, the auxiliary sheet itself for the absorbent article has also the advantage of having a preferred hardness that does not damage the wearing feeling.

As adhesives, water-soluble polymers, crosslinking agents, plasticizers, gel adhesives composed of water content, or the like can be cited. To be more specific, as examples of the water-soluble polymers, gelatin, sodium poly acrylate, polyvinyl alcohol, carboxyl methyl cellulose, or the like, and as examples of the crosslinking agents, water soluble metal salt such as calcium chloride, magnesium sulfate, and as examples of the plasticizers, glycerin, wax, paraffin or the like can be cited.

In addition to the above, a pressure-sensitive hot melt adhesive can be used. This pressure-sensitive hot melt adhesive comprises essentially of a synthetic rubber resin such as styrene-butadiene-styrene block copolymer (SBS), styrene-isoprene-styrene block copolymer (SIS), styrene-ethylene/butadiene-styrene block copolymer (SEBS), and styrene-ethylene/propyrene-styrene block copolymer (SEPS), and can be obtained by fusing and mixing a tackifier such as a terpene resin and a rosin resin and a plasticizer such as wax with the above.

Foam melt is obtained by blowing air or nitrogen gas into a pressure-sensitive hot melt adhesive when the pressure-sensitive hot melt adhesive is fused, forming lots of minute bubbles inside by expansion of the air or nitrogen gas blown therein, and making it foamy with a magnification of 3 to 20 times. The foam melt is also effective because of its rich elasticity.

Further, as the other adhesives, silicone type adhesives can be used. As an example of the silicone type adhesives, a mixture essentially comprising of silicone resin and fluorocarbon polymers resin mixed with a crosslinking agent of metal salt such as platinum, molybdenum, and antimony, and a plasticizer such as ester type wax, glycerin, and machine oil, can be cited.

Although many adhesives exist for forming the adhesive portion as the above, the use of a pressure-sensitive hot melt adhesive is preferred considering application stability. To be more specific, an adhesive fusing and mixing 15 to 25 weight % of SEBS, 15 to 35 weight % of plasticizer, and 40 to 70 weight % of tackifier is preferred for the use. To this pressure-sensitive hot melt adhesive, an oxidation inhibitor, a fluorescence inhibitor, or the like can also be added in the range of 0.1 to 1.0 weight %.

As a preferred example of an adhesive portion, a composite adhesive portion, which is applied with an adhesive in a spiral form and sprayed on the front surface applied with form melt, can be cited. To be more specific, such an adhesive portion as a low adhesion type adhesive obtained by mixing and fusing 25 to 50 weight % of SEBS, 25 to 40 weight % of plasticizer, and 10 to 50 weight % of tackifier is mixed with nitrogen gas, and said adhesive is applied to the front surface of the foam adhesive portion in the range of 20 to 100 g/m² at a foaming magnification of 5 to 15 times, can be cited. In such a manner, the adhesive portion can be made comfortable and excellent in rubber elasticity. Moreover, the adhesive portion may be arranged by cutting the adhesive into tape-like pieces and then sticking them thereon.

The adhesive portion is preferred to be pre-coated with a sheet made by coating a sheet of thin paper generally used for separate paper with a silicone resin, or pre-coated with a sheet made by coating a plastic film with a silicone resin. Thus, the adhesive portion can be prevented from being polluted and peeled off during storage.

(8) The auxiliary sheet for the absorbent article according to (7), wherein said adhesive portion is formed in a shape of a belt passing in the vicinity of a circumferential portion of said through-hole, or said adhesive portion is formed along said circumferential portion thereof.

In the auxiliary sheet for the absorbent article in accordance with the present invention, the adhesive portion is arranged in the vicinity of the through-hole or the periphery thereof. Therefore, the adhesion of labia to the through-hole is enhanced, and diffusion of the body fluid (especially, menstrual blood) flowing out of labia can be prevented better. Moreover, when the position of the through-hole is biased as in the case of (6) stated above, it is preferable to bias also the position of the adhesive portion associated therewith.

Especially, to prevent the adhesive from adhering to pubic hairs which are present in front of the labia, a belt-like adhesive portion is preferred to be arranged in the vicinity of both peripheries of the through-hole, and preferably, the arrangement is in the range of 1 to 5 mm.

(9) The auxiliary sheet for the absorbent article according to (7) or (8), further comprising an elastic sheet on a body side face of the auxiliary sheet for the absorbent article, and said adhesive portion is placed on the elastic sheet.

According to the auxiliary sheet for the absorbent article in accordance with the present invention described above, the adhesive portion is provided on an elastic sheet. Since such an elastic sheet has a bending resistant property with repulsive feeling and a form recovering property, a wearer is able to perform wearing operation in the state of maintaining the first form of wearing. Therefore, when affixing the auxiliary sheet for the absorbent article on the skin, it is possible to prevent the adhesives from adhering to each other and causing wrinkles inconvenient for putting it on. As a result, it becomes possible to affix the adhesive portion closely to the body, and prevent such a situation as the wearing feeling is damaged by wearing it in the wrinkled state.

It is preferable that the adhesive portion is placed so as to be divided on the right and left at 1 to 10 mm intervals in the neighborhood of the front and rear ends bordered by the longitudinal central line. In such a manner, the auxiliary sheet for the absorbent article moves following up the movement of a wearer, and this follow-up movement can prevent the skin affixed with the auxiliary sheet for the absorbent article from being twitched in the adhesive portion. As a result, the feeling to wear becomes suitable, and moreover, departure of the auxiliary sheet for the absorbent article adhered to the body can be reduced. Moreover, instead of the divided arrangement of the adhesive portion as the above, it may be provided with long grooves which will described later.

(10) The auxiliary sheet for the absorbent article according to (9), wherein said auxiliary sheet for the absorbent article is provided with a plurality of long grooves having a recess shape in parallel in the longitudinal direction in a range where said elastic sheet is placed.

According to the auxiliary sheet for the absorbent article in accordance with the present invention as the above, within the range where an elastic sheet is provided, a plurality of recessed form grooves is arranged side by side in the longitudinal direction to form bellows. Therefore, the bellows expands in the lateral direction following up the movement of a wearer, and are able to prevent the skin from being twitched, which is adhered to the auxiliary sheet for the absorbent article in the adhesive portion.

The long grooves mentioned above can be formed by performing mechanical processing by corrugation in the longitudinal direction of the auxiliary sheet for the absorbent article. Such long grooves are preferred to be formed within the width range of 1 to 10 mm.

(11) The auxiliary sheet for the absorbent article according to any one of (1) to (10), further comprising a body fluid guiding stem for guiding a body fluid from said through-hole to said sheet-form absorbent article.

According to the auxiliary sheet for the absorbent article in accordance with the present invention, the through-hole arranged in the auxiliary sheet for the absorbent article is tied to a body fluid guiding stem projecting from the body side toward the opposite side to the body side. Therefore, the body fluid (especially, menstrual blood) is surely made to flow down to the sheet-form absorbent article along the body fluid guiding stem from the through-hole, and the body fluid can surely be led to the sheet-form absorbent article.

The body fluid guiding stem can integrally be formed, for example, by partly folding the auxiliary sheet for the absorbent article from the through-hole part to be extended out downward, and it can be formed by deforming a separate sheet from the auxiliary sheet for the absorbent article into a form suitable for the body fluid guiding stem and attaching it thereto.

The form of the body fluid guiding stem is preferred to be cylindrical almost like an unbroken continuous tunnel. By making it into such a form, the body fluid can be made to smoothly flow into the sheet-form absorbent article. Moreover, the shape of vertical cross-section of the body fluid guiding stem is not to be specifically restricted to a cylindrical form, a conical form, an inverse-conical form, or the like, unless the body fluid flow is obstructed.

When the body fluid guiding stem is formed from a separate sheet, the opening on the body side of the body fluid guiding stem is preferred to be equivalent to the through-hole in size, to be more specific, it is preferred that the longitudinal dimension is made to 90 to 140 mm, and the lateral dimension is made to 35 to 70 mm. Moreover, the dimension of the height is preferred to be 10 to 40 mm. By forming the body fluid guiding stem in such dimensions, it is possible to guide the body fluid into the sheet-form absorbent article surely and also within the minimum range.

For the sheet to be formed into the body fluid guiding stem, a material excellent in flexibility and bulkiness is preferable for the use. Moreover, the body fluid guiding stem can be formed from a single material, however, it is preferred to be formed from a composite material having a hydrophilic property inside and a hydrophobic property outside. This is because the situation of the body fluid flowing outside of the body fluid guiding stem is relaxed by covering the inside hydrophilic material by the outside hydrophobic material, as a result, the flow of the body fluid can surely be oriented to the sheet-form absorbent article to be used together.

As a material which can be used for such a body fluid guiding stem, a fiber sheet such as spun bond, melt blown, through-air, point bond, and airlaid, or a single or composite material of a film, a three-dimensionally perforated film, an air permeable film, or a foam sheet or the like can be cited.

When the body fluid guiding stem is formed of a single material, for example, in the case of a fiber sheet unifying double-layered fiber webs, it is preferable to use the webs, of which the front surface is formed of a single or a sheath-core structured hydrophobic fiber of 1.1 to 3.3 dtex in the range of 10 to 25 g/m² and the rear surface is formed of the fiber of 1.1 to 2.2 dtex having a similar structure and provided with hydrophilic treatment in the range of 15 to 35 g/m², by sheeting them by means of the through-air method. To be more specific, this is a bicomponent synthetic fiber of the sheath-core structure of PE/PP and a 2.6 mm sheet of fiber obtained by unifying the fiber webs of which the front surface is formed of 2.2 dtex thick fiber by a specific weight per unit of 15 g/m², and of which the rear surface is formed of the bicomponent synthetic fiber provided with hydrophilic treatment and having 1.6 dtex thickness by a specific weight per unit of 25 g/m², and sheeting them by the through-air method.

As an example of another material when the body fluid guiding stem is formed of a single material, a foam material having a closed cell structure can be cited. To be more specific, there is a preferable form material of 1.5 mm in thickness obtained by foaming a resin of PE/EVA mixed in the ratio of 80 to 20 at a magnification of 15 times and then cross-linking them by irradiating it with electrons, cutting it in half in the direction of thickness, making the front side as a skin layer and the inner side as a cell layer, and coating the inner side with a surface active agent.

As a material formed of a composite material, a material made into a fiber sheet, of which for the front surface side is mixed melt blown nonwoven fabric and for the inner surface side is made a mixture of hydrophilic fiber, can be cited as a preferred example. To be specific, such a material can be cited, as for the front surface side is mixed melt blown nonwoven fabric of PP of 15 to 30 g/m², and for the inner surface side is mixed nonwoven fabric of 20 to 40 g/m² obtained by fiber-blending 2.2 dtex bicomponent synthetic fiber of the PE/PP sheath-core structure provided with hydrophilic treatment with 1.5 dtex reproduced cellulose in the ratio of 70 to 30 by weight and by means of a water-flow interlacing treatment, and further a joint area rate of both nonwoven fabric is made to 13%. To be more specific, the blown nonwoven fabric is composed by 25 g/m², and the woven fabric sheet obtained by water-flow interlacing is composed by 25 g/m².

As another example when the body fluid guiding stem is formed of a plurality of materials, such a body fluid guiding stem can be cited, as the front surface side is composed of a three-dimensional foam film, and melt blown nonwoven fabric provided with hydrophilic treatment is stuck on the inner surface side. To be specific, the foam film comprises essentially of LDPE resin of 0.915 g/cm³ density containing 5% titanium oxide; the specific weight per unit is adjusted to 20 g/m²; the cross-sectional form is tapered; the surface opening rate is 55%; and the hole size is 0.28 mm. This foam film is sprayed with melt blown fiber of 25 g/m² comprising PP in the molten state on the inner surface, or a sheeted foam film is stuck on inner surface by heat-embossing. The melt blown fiber or the fiber sheet is sprayed or coated with a surface active agent for providing it with hydrophilic treatment.

(12) The auxiliary sheet for the absorbent article according to (11), wherein said body fluid guiding stem has a bottom formed on the opposite side to the body side.

According to the auxiliary sheet for the absorbent article in accordance with the present invention, the body fluid guiding stem placed on the opposite body side face to body side face is formed to have a bottom. Therefore, the body fluid (especially, menstrual blood) guided into the body fluid guiding stem is temporarily stored therein. As result, even when a large amount of the body fluid is excreted and the amount exceeds liquid permeable capability of the surface of the sheet-form absorbent article located on the opposite side to the body side of the auxiliary sheet for the absorbent article, the body fluid is prevented from leaking outside of the sheet-form absorbent article.

Thus, by using the sheet-form absorbent article together with the auxiliary sheet for the absorbent article in accordance with the present invention, the sheet-form absorbent article is enhanced in the fluid permeable adaptability.

Moreover, in the present invention, the bottom shape and the opening portion shape of the body fluid guiding stem are not necessarily of the same shape. For example, they may be formed into a conical form gradually increased in the bottom shape and also formed into a form of a bowl gradually decreased in the bottom shape.

(13) The auxiliary sheet for the absorbent article according to any one of (1) to (12), further comprising a belt-like fixing tab for fixing the auxiliary sheet on said sheet-form absorbent article at both sides of the longitudinal direction of said auxiliary sheet for the absorbent article are provided with.

The auxiliary sheet for the absorbent article in accordance with the present invention as the above is provided with belt-like fixing tabs on both longitudinal sides to be fixed to the sheet-form absorbent article. Therefore, a wearer picks up such fixing tabs and wraps them around the sheet-form absorbent article, and thereby the wearer is able to fix the auxiliary sheet for the absorbent article to the sheet-form absorbent article. Thus, according to the present invention, it becomes possible to unify the auxiliary sheet for the absorbent article with the sheet-form absorbent article, therefore, it is possible to make the sheet-form absorbent article surely secure the body fluid (especially, menstrual blood) against diffusing and also prevent a rough dry skin.

The dimensions of the fixing tabs are preferred to be in the range of 15 to 35 mm in the longitudinal direction, and 10 to 20 mm in the lateral direction. With these dimensions, the operability is favorable, and also they can be long enough to fix the auxiliary sheet for the absorbent article to the sheet-form absorbent article.

As the sheet-form absorbent article for fixing thereto the auxiliary sheet for the absorbent article in accordance with the present invention, a thin type sanitary napkin of 1 to 5 mm thickness is preferred for the use. Such a thin type sanitary napkin gives little feeling of wear and provides comfort to a wearer, while it is prone to cause a gap between labia and the thin type sanitary napkin, therefore, the sanitary napkin is in inherent danger of causing lateral leakage of the body fluid leaking down on the skin of the wearer. Concerning this point, when the auxiliary sheet for the absorbent article in accordance with the present invention is laid between labia and the thin type sanitary napkin, the gap is not caused but the body fluid is oriented to flow in a specific direction, therefore, the body fluid becomes resistant to lateral leaks. Thus, by using the auxiliary sheet for the absorbent article in accordance with the present invention together when using the thin type sanitary napkin, it is possible for a wearer to reduce the danger of the body fluid leakage while enjoying the comfort.

(14) The auxiliary sheet for the absorbent article according to any one of (1) to (13), wherein said auxiliary sheet for the absorbent article is an auxiliary sheet for the absorbent article used for incontinence of urine.

According to such auxiliary sheet for the absorbent article of the present invention, it can be used for incontinence. In short, as the ostium vaginae discharging menstrual blood and the urethral meatus for discharging urine are both found between interlabia, urine can be absorbed in the absorbent article as urine discharged from the urethral meatus is guided to the absorbent article, in case of using the auxiliary sheet for the absorbent article according to the present invention in a manner its through-hole are engaged in the labia.

Thus, according to the present invention, the auxiliary sheet is effective for incontinence, as it can absorb urine without diffused.

(15) The auxiliary sheet for the absorbent article according to any one of (1) to (13), wherein said auxiliary sheet for the absorbent article is an auxiliary sheet for the absorbent article used for absorbing a vaginal discharge.

Such auxiliary sheet for the absorbent article of the present invention can be used for absorbing surely the vaginal discharge into the absorbent article. In short, as the auxiliary sheet for the absorbent article according to the present invention is used in a manner its through-hole are engaged in the labia, it can guide also the discharge (vaginal discharge) other than menstrual blood to the absorbent article, so it can be used for this application (vaginal discharge absorption).

Thus, according to the present invention, the auxiliary sheet is effective for a wear without at the period of menstruation, as it can absorb the vaginal discharge speedy to reduce her discomfort.

### BRIEF DESCRIPTION OF THE DRAWINGS

FIG. 1 is a schematic perspective view showing an auxiliary sheet for an absorbent article according to this embodiment;

FIG. 2 is an explanatory illustration explaining a contact state between a sanitary napkin and labia;

FIG. 3 is an illustration showing a fixed state of the auxiliary sheet for the absorbent article according to this embodiment;

FIG. 4 is a schematic perspective view showing the auxiliary sheet for the absorbent article in which a through-hole is located near the rear end portion in the longitudinal direction, and is an example of deformed auxiliary sheet for the absorbent article according to this embodiment;

FIG. 5 is a schematic perspective view showing the auxiliary sheet for the absorbent article of which the front area is formed wider, and is an example of deformed auxiliary sheet for the absorbent article according to this embodiment;

FIG. 6 is an explanatory illustration explaining a two-layer structure comprising a soft touch sheet and a liquid impermeable sheet, showing a vertical sectional structure of the auxiliary sheet for the absorbent article according to this embodiment;

FIG. 7 is a vertical sectional view explaining a three-layer structure holding a liquid impermeable sheet between soft touch sheets, showing another vertical sectional structure of the auxiliary sheet for the absorbent article according to this embodiment;

FIG. 8 is a schematic perspective view showing the auxiliary sheet for the absorbent article provided with an elastic sheet on the body side face and with an adhesive portion on the elastic sheet, and is an example of deformed auxiliary sheet for the absorbent article according to this embodiment;

FIG. 9 is a schematic perspective view showing the auxiliary sheet for the absorbent article provided with a plurality of long recessed form grooves in column in parallel in the longitudinal direction within the range in which the elastic sheet is attached, and is an example of deformed auxiliary sheet for the absorbent article according to this embodiment;

FIG. 10 is a vertical sectional view of the auxiliary sheet for the absorbent article which is partly extended to the sanitary napkin side and formed into a cylindrical body fluid guiding stem, and is another embodiment of the auxiliary sheet for the absorbent article according to this embodiment;

FIG. 11 is an explanatory illustration explaining a state in which the opposite body side face to body side face of the body fluid guiding stem, which is formed as a part of the auxiliary sheet for the absorbent article, is in contact with the sanitary napkin;

FIG. 12 is a vertical sectional view from the front showing a fixed state of the auxiliary sheet for the absorbent article of which is partly formed into the body fluid guiding stem;

FIG. 13 is a vertical sectional view of the auxiliary sheet for the absorbent article provided with the body fluid guiding stem having a bottom;

FIG. 14 is a schematic perspective view showing the auxiliary sheet for the absorbent article according to this embodiment, provided with belt-like fixing tabs;

FIG. 15 is a schematic perspective view showing a state in which the auxiliary sheet for the absorbent article according to this embodiment is fixed on a sanitary napkin by the fixing tabs;

FIG. 16 is a schematic perspective view showing a state in which the auxiliary sheet for the absorbent article according to this embodiment is fixed on a sanitary napkin of another shape by the fixing tabs.

### DETAILED DESCRIPTION OF THE PREFERRED EMBODIMENT

Next, an embodiment of the auxiliary sheet for the absorbent article in accordance with the present invention will be explained below referring to the drawings.

[Basic Configuration]

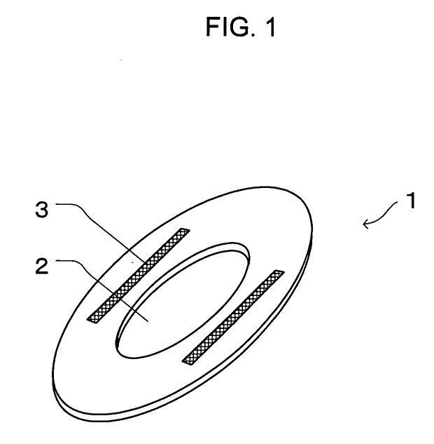

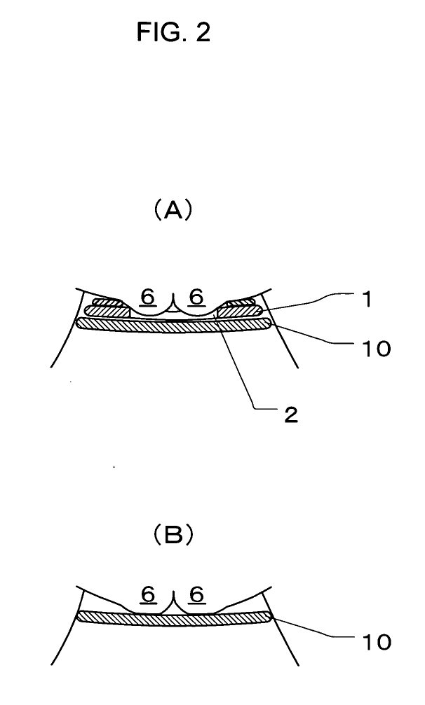

Firstly, the configuration of the auxiliary sheet for the absorbent article of this embodiment will be explained. FIG. 1 illustrates a schematic perspective view of the auxiliary sheet for the absorbent article 1 (hereafter, called an auxiliary sheet) in this embodiment, and FIG. 2 is a longitudinal section viewing from the front for explaining a contact state between the sanitary napkin 10 and labia 6.

The auxiliary sheet 1 in the mode of this embodiment is to be used by putting it on the body side face of the sheet-form absorbent article such as a sanitary napkin, and is to be used for preventing the body fluid (especially, menstrual blood) absorbed in the sanitary napkin or the like from coming into direct contact with the skin of a wearer. Based on such a function, the auxiliary sheet 1 is smaller than the sanitary napkin in size, and also its appearance is substantially vertically long elliptic so as to conform to the inside of the thigh of the wearer who wears it. As shown in FIG. 1, the auxiliary sheet 1 is provided with an elliptic through-hole 2 in the central portion, and an adhesive portion 3 in the neighborhood of the circumference of the through-hole on the body side face. Since the through-hole 2 is large enough to naturally put oblong elliptic labia therein, the wearer is easily able to fit a pair of labia in the through-hole 2. Moreover, since the through-hole 2 is formed oblong elliptic as described above, menstrual blood flowing along the labia which are generally longitudinal shape is surely guided from the through-hole 2 to the sanitary napkin which is not illustrated here. Therefore, it is possible to reduce direct contact between the menstrual blood and the skin, and also reduce the menstrual blood in widely remaining and diffusing. Thus, it is possible to reduce a rough dry skin while wearing the sanitary napkin.

As to the outside dimensions of the auxiliary sheet 1, the longitudinal dimension is preferred to be in the range of 80 to 200 mm in the longitudinal direction, more preferably, in the range of 130 to 180 mm. Moreover, the lateral direction is preferred to be in the range of 40 to 90 mm, more preferably, in the range of 50 to 70 mm. With such dimensions, the auxiliary sheet can be sized suitable for being fixed on the sanitary napkin or the like.

In the case of fixing the auxiliary sheet in accordance with the present invention, as shown in FIG. 2(A), the labia 6 are engaged in the through-hole 2. Therefore, differently from the case in which the sanitary napkin 10 as shown in FIG. 2(B) is singly used, the labia 6 hardly come into contact with the menstrual blood absorbed into the sanitary napkin 10, and a rough dry skin can be prevented. Moreover, by increasing the auxiliary sheet 1 in thickness, it is possible to secure non-contact between the labia 6 and the sanitary napkin 10.

[An Example of a Deformed Through-Hole]

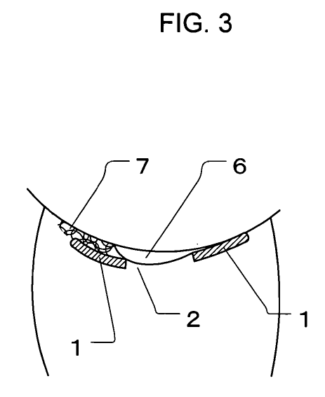

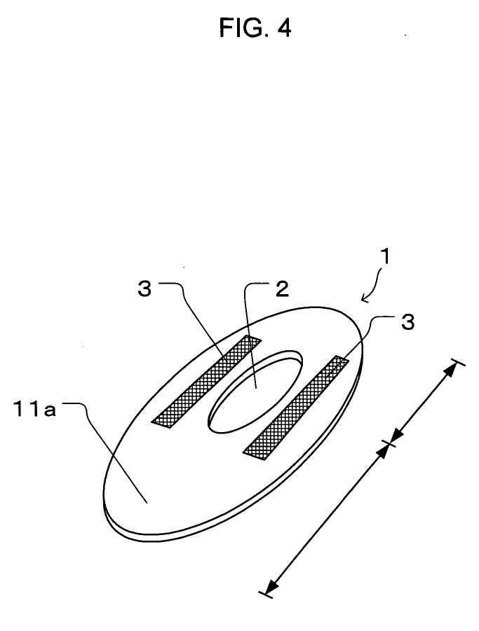

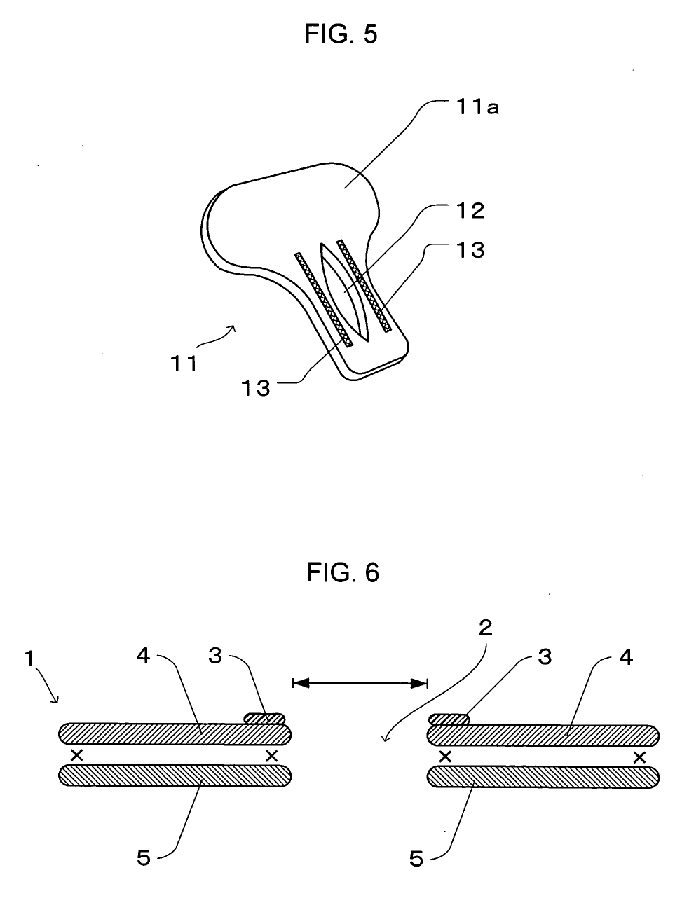

Next, an example of a deformed through-hole of this embodiment will be explained. FIG. 3 is a longitudinal section showing the state in which the fixed state of the auxiliary sheet 1 provided with the through-hole in the center according to this embodiment is laterally viewed against the body; FIG. 4 illustrates the auxiliary sheet 1 in which the through-hole 2 is biased backward; and FIG. 5 shows the auxiliary sheet 11 in which the through-hole 12 is biased backward and also the front area 11a is laterally widen.

As shown in FIG. 3, in general, there is a pubic hair portion 7 in the front area (near pubis) of the labia where lots of pubic hairs exist, therefore, the pubic hairs impede close adhesion of the auxiliary sheet 1 to the body in such a pubic hair portion 7, and sometimes cause a gap in-between. Moreover, the menstrual blood is also in danger of diffusing via the pubic hairs lying in the gap.

Therefore, the leakage of the menstrual blood can be reduced by locating the through-hole 2 not in the central portion as that in the auxiliary sheet 1 shown in FIG. 1, but locating the through-hole 2 near the rear end portion of the auxiliary sheet 1 as shown in FIG. 4, elongating the front area 11a coming into contact with the pubic hair portion when fixing the sheet, and thereby receiving the diffused menstrual blood in the front area 11a. Moreover, it is preferable to bias the location of the adhesive portion 3 accompanied by biasing the location of the through-hole 2, to prevent the adhesive from adhering to the pubic hairs.

Further, as shown in FIG. 5, not only the locations of the through-hole 12 and the adhesive portion 3 in the auxiliary sheet 11 are biased backward, but also the front area 11a can be formed wider. With such a form, the area where the auxiliary sheet 11 comes into contact with the pubic hairs is enlarged, and even when the menstrual blood leaking from the inside of the labia come into contact with the pubic hair portion and diffuses, the front region 11a having a wide area can receive the diffusion.

[Vertical Section Structure of Auxiliary Sheet]

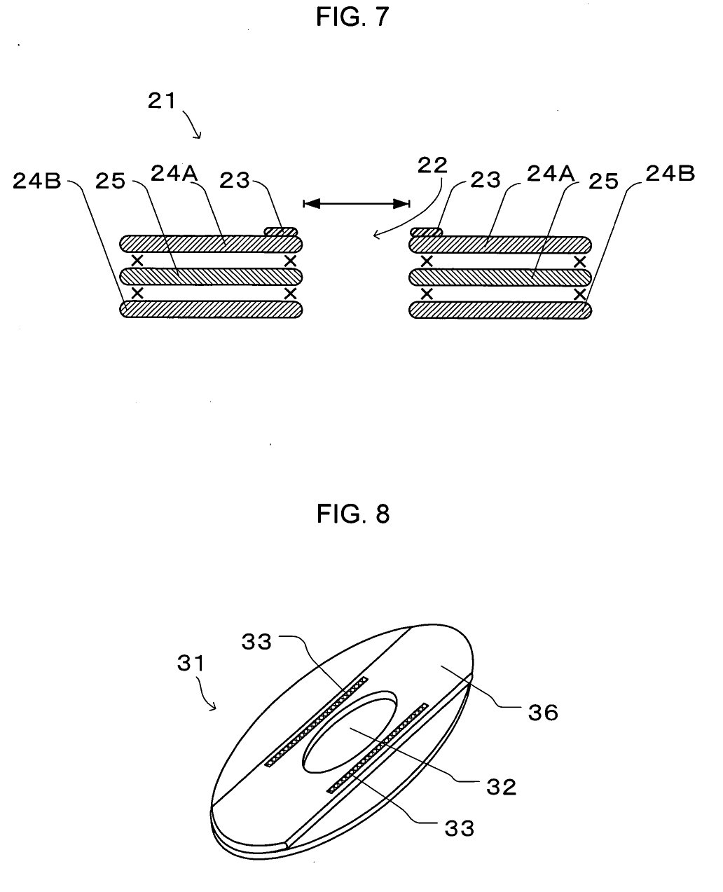

Next, the vertical section structure of the auxiliary sheet 1 viewed from the front will be explained below. FIG. 6 shows the auxiliary sheet 1 of this embodiment, and is a vertical sectional view for explaining the two-layer structure comprising a soft touch sheet 4 and a liquid impermeable sheet 5, and FIG. 7 shows another sectional view of the auxiliary sheet 1 in this embodiment, and is a vertical sectional view for explaining an auxiliary sheet 21 comprising a three-layer structure holding a liquid impermeable sheet between soft touch sheets 24A and 24B.

As shown in FIG. 6, the auxiliary sheet 1 in this embodiment is of a two-layer structure in which the soft touch sheet 4 is placed on the side contacting the skin, and the liquid impermeable sheet 5 is placed on the side contacting the sanitary napkin, and these are jointed with an adhesive or by thermal sealing. The soft touch sheet 4 gives comfort to wear, while the liquid impermeable sheet 5 is able to prevent re-contact between the re-wetting menstrual blood due to pressure of wearing and the skin.

Moreover, as an auxiliary sheet 21 shown in FIG. 7, it can also be made into a three-layer structure by sandwiching the liquid impermeable sheet 25 between the soft touch sheet 24A placed on the side contacting the skin and the soft touch sheet 24B placed on the side contacting the sanitary napkin. In such a structure, the soft touch sheet 24A contacting the skin gives a comfortable wearing feeling to wearer, and the soft touch sheet 24B contacting the sanitary napkin absorbs the re-wetting menstrual blood and prevents the menstrual blood from flowing.

[Example of Deformed Adhesive Portion]

Next, an example of a deformed adhesive portion 3 of the auxiliary sheet 1 in this embodiment will be explained below. FIG. 8 is a schematic perspective view showing an auxiliary sheet 31 provided with an elastic sheet 36 thereon and provided with an adhesive portion 33 on the elastic sheet 36. FIG. 9 is a schematic perspective view showing an auxiliary sheet 41 provided with a plurality of long recessed form grooves 47 in column in parallel in the longitudinal direction within the range in which an elastic sheet 46 is attached.

The adhesive portion can be arranged directly on the body side face of the auxiliary sheet, however, it can be provided with elasticity in order to prevent the adhesives from adhering to each other. For example, as shown in FIG. 8, an elastic sheet 36 can be mounted on the zone which covers the through-hole 32 and two adhesive portions 33 arranged in the periphery of the through-hole 32 in the auxiliary sheet 31. In such a manner, the adhesive portions 33 are provided with elasticity, and this facilitates fixing operation by a wearer.

Moreover, when the elastic sheet 36 is placed on the whole longitudinal area of the auxiliary sheet 31 as in this embodiment, the portion corresponding to the through-hole 32 is hollowed to the same size as the through-hole 32 from the elastic sheet 36 in advance.

Moreover, as shown in FIG. 9, when a plurality of recessed form long grooves 47 is arranged in parallel in the longitudinal direction into bellows in the range where the elastic sheet 46 is placed, the auxiliary sheet 41 is widened in the width in the lateral direction according to the movement of a wearer and is deformed flat. Therefore, the skin stuck with the adhesive is prevented from being twitched as the wearer moves. Moreover, the long grooves 47 are formed by mechanical corrugating processing.

[Body Fluid Guiding Stem]

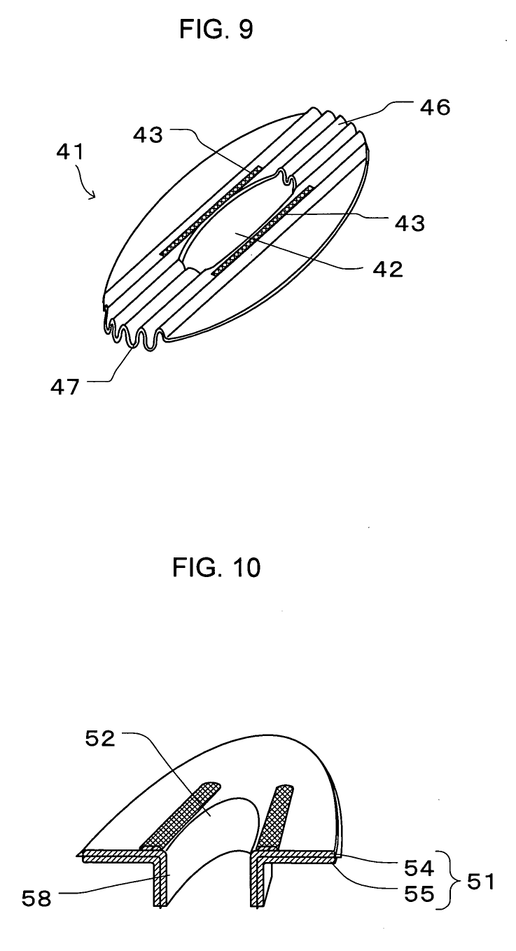

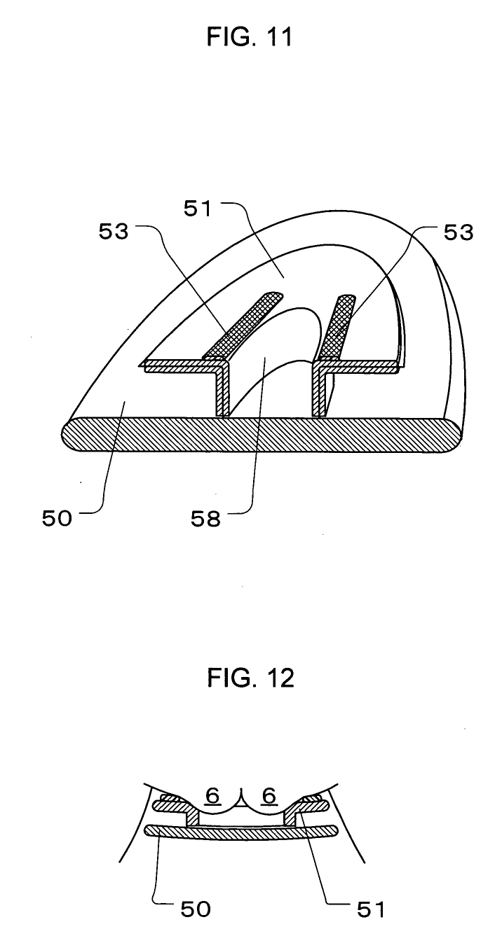

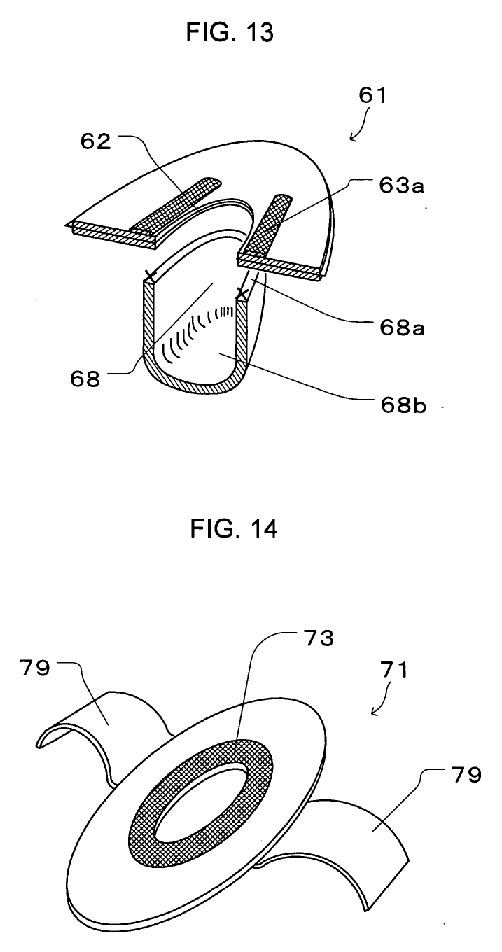

Next, the body fluid guiding stem will be explained below. FIG. 10 is a vertical sectional perspective view of an auxiliary sheet 51 which is partly extended to the opposite side to the body side and formed into a cylindrical body fluid guiding stem 58; FIG. 11 is an explanatory drawing for explaining the state in which the opposite body side face to body side face of the body fluid guiding stem 58 is in contact with the sanitary napkin 50; FIG. 12 is a vertical section viewed from the front showing the fixed state of the auxiliary sheet 51; and FIG. 13 is a vertical sectional view of an auxiliary sheet 61 mounted with a body fluid guiding stem 68 having a bottom on the plane of the opposite side to the body side.

In order to prevent menstrual blood from diffusing more effectively, the auxiliary sheet 1 related to this embodiment can be provided with a body fluid guiding stem. For example, as shown in FIG. 10, a portion of the auxiliary sheet 51 formed of a two-layer structure comprising a soft touch sheet 54 and a body liquid impermeable sheet 55 is bent from a through-hole 52 portion and extended downward, and the body fluid guiding stem 58 is formed in such an extended portion. In such a structure, the menstrual blood leaking from the inside of the labia engaged in the through-hole 52 and the body fluid guiding stem 58 tied thereto flows toward the sanitary napkin (not shown) in the body fluid guiding stem 58, therefore, the menstrual blood is effectively prevented from remaining in and diffusing to the vicinity of the labia. Moreover, since the body fluid guiding stem 58 is formed in one body with the auxiliary sheet 51 in this embodiment, both are tightly coupled, and this permits to facilitate a manufacturing process by omitting a bonding process.

The auxiliary sheet 51 is fixed on the inside of the thigh of a wearer by the adhesive portion 53, and the sanitary napkin 50 is fixed on the underwear. Then, as shown FIG. 11, when putting on underwear, the body fluid guiding stem 58 of the auxiliary sheet 51 comes into contact with the sanitary napkin 50.

Moreover, the opposite body side face to body side face of the body fluid guiding stem 58 can be adhered to the body side face of the sanitary napkin 50 with an adhesive. In such a manner, it is possible to fix the body fluid guiding stem 58 at a suitable position on the sanitary napkin 50, therefore, it is possible to prevent the body fluid guiding stem 58 from being dislocated accompanied by the movement of the wearer.

As shown in FIG. 12, in the case of fixing the auxiliary sheet 51 in this embodiment, the labia 6 can be secured for non-contact with the sanitary napkin 50 surer than in the case of FIG. 2(A) described above.

Moreover, as shown in FIG. 13, the body fluid guiding stem 68 having a bottom can also be formed by separately mounting a sheet which is formed so as not stick out of the outer outline of the auxiliary sheet 61, and has an opening portion 68a of a form at least equivalent to the through-hole 62. In such a case, even when a discharge amount of menstrual blood exceeds the liquid permeable capability of the sanitary napkin (not shown), the menstrual blood is temporarily stored in the body fluid guiding stem 68 because the body fluid guiding stem 68 has a bottom 68b, and the liquid permeable capability of the sanitary napkin is increased in adaptability.

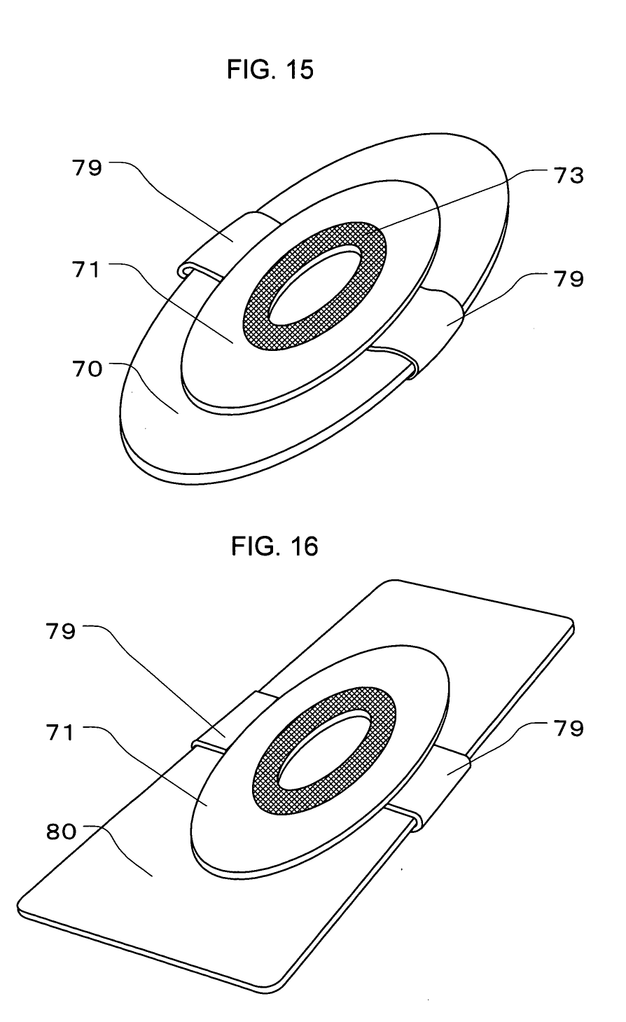

Next, an auxiliary sheet provided with belt-like fixing tabs will be explained below. FIG. 14 is a schematic perspective view showing the auxiliary sheet 71 provided with the fixing tabs 79, and FIG. 15 is a schematic perspective view showing the state in which the auxiliary sheet 71 is fixed on a sanitary napkin 70 by the fixing tabs 79. FIG. 16 is a schematic perspective view showing the state in which the auxiliary sheet 71 is fixed on a sanitary napkin 80 of another shape.

In this embodiment, as shown in FIG. 14, the fixing tabs 79 allowing a wearer to pick them up by fingers are provided on both of the longitudinal side portions of the auxiliary sheet 71 so as to be extended from both of the right and left side edges. According to this structure, as shown in FIG. 15, it becomes possible to tightly fix the auxiliary sheet 71 on the sanitary napkin 70 by wrapping these fixing tabs 79 around the sanitary napkin 70.

Moreover, although an adhesive portion 73 is provided around the through-hole on the auxiliary sheet 71 in this embodiment, the adhesive portion may be omitted considering the operability at the time of fixing.

In this embodiment, the sanitary napkin 70 is almost elliptical in shape as well as the auxiliary sheet 71, however, the sanitary napkin 70 is not to be restricted to this shape, but may be rectangular like a sanitary napkin 80 for general use shown in FIG. 16.

[Biodegradable, Water Dispersible, and Water Soluble Material]

The auxiliary sheet in accordance with the present invention can employ materials and compositions which can be processed by flushing the toilet to perform an after-use disposal process simply and sanitarily. For that purpose, the auxiliary sheet need to be made of such materials as have biodegradability and/or water dispersibility and/or water solubility.

“Biodegradability” means that a material is degraded into a gas such as carbon dioxide or methane or the like, water, and biomass under the anaerobic or aerobic condition according to the process of the nature under the existence of germs, bacteria, ray fungi, and other microbes, and that the performance of biodegradation (a biodegradable rate, a biodegradable degree, etc.) is compared to that of natural materials such as fallen leaves, or compound polymers generally recognized biodegradable in the same environment.

“Water dispersibility” has the same meaning of a hydrolyzable property, and means such a property as fibers are not influenced by a limited quantity of water (menstrual blood) when used, but are easily dispersed from each other into small pieces in a large quantity of water or water current at least to the extent that ordinary toilette piping is not clogged therewith. “Water solubility” means a property that although the material is not influenced in a limited quantity of water (menstrual blood) when used, it dissolves in a large quantity of water or water current.

The material is not to be specially restricted as long as the above-mentioned conditions are satisfied, however, the following materials can be cited as examples.

As fibers to be used for a liquid permeable material, natural fibers and/or chemical fibers can be used. As examples of the natural fibers, tissue, pulverized pulp, airlaid pulp formed from chemical bonding by water soluble resin, cotton, or the like can be cited. As examples of the chemical fibers, recycled cellulose of rayon, fibril rayon, or the like; as examples of the synthetic fibers, synthetic fibers or the like provided with hydrophilic treatment to PE, PP, PET, ethylene-vinylacetate copolymer, or the like; as examples of synthetic biodegradable fibers, poly lactic acid, polybutylene succinate, or the like; as examples of the materials having water solubility, carboxymethyl cellulose, polyvinyl alcohol, or the like, can be cited. Among said materials, the natural fibers such as pulp and cotton, rayon, poly lactic acid, or the like having biodegradability are preferred for the use.

Moreover, said materials can be used singly or mixed, or formed into a web or nonwoven fabric. Web-forming of what is called biodegradable fibers such as poly lactic acid and polybutylene succinate are carried out by either a dry process or a wet process by means of a card method, a spun bond method, a melt blown method, or an airlaid method, or may be carried out by combining two or more of these methods. As bonding methods, the methods such as thermal bonding, needle punch, chemical bonding can be cited, however, the bonding is not specially to be restricted to these methods. Moreover, spun lace processed into a sheet form by the water-flow interlacing treatment can be used.

As examples of forming methods for providing water dispersibility, a method for forming fibers into a sheet form of water dispersible paper by hydrogen-bonding fibers with each other, a method for forming fibers into a sheet form of water dispersible paper by bonding the fibers with each other with a water soluble binder, or a method for forming fibers into a sheet form of water dispersible paper by confounding the fibers, can be cited.

Moreover, to provide the fibers with a favorable water dispersibility, a fiber length is preferred to be in the range of 2 to 51 mm, more preferably, in the range of 2 to 10 mm. Further, in order to let the fibers own both of the water dispersibility and non-damageable strength when used, it is preferable to select the fineness (thickness) from the range of 1.1 to 3.3 dtex. Within this range, the water dispersiblity is favorable, however, the fibers are prone to become fluffy and lose, and exceeding this range on the other hand, the fibers are remarkably decreased in water dispersibility.

As the liquid impermeable materials, cellulose derivatives such as methyl cellulose, hydroxy ethyl cellulose, carboxy methyl cellulose; water-soluble polymers such as polyvinyl alcohol, sodium alginate, sodium poly acrylate, polyacrylate ether, polyvinyl pyrrolidone, and copolymer of isobutylene and maleic anhydride; or biodegradable polymers such as poly lactic acid, polybutylene succinate, starch, and dextrin, can be cited.

As to the materials, a single material or a mixture of the materials can also be formed into a film sheet. Further, the film sheet may be coated with a water repellant material such as a silicone resin, or both may be mixed, and nonwoven fabric made of the material(s) may be processed by laminating.

<Bonding Method>

To join the sheets, bonding methods such as bonding by using polyvinyl alcohol having water solubility or water-swelling property, heat sealing, or hydrogen bonding are adopted singly or by a suitable combination of them.

<Elastic Material>

As a material for providing the auxiliary sheet with elasticity, natural rubber (cis-1, 4-polyisoprene) can be cited as an example of a material having biodegradability. The natural rubber is considered to follow the degradation processes such as successive chain splitting and to decrease a molecular weight by facilitating an attack to the rubber by any of oxygen and/or bacteria or germs. Or the material may be water soluble elastomer or the like.

### INDUSTRIAL APPLICABILITY

The auxiliary sheet for the absorbent article in accordance with the present invention as the above remarkably reduces a contact range between the sheet-form absorbent article and the body of a wearer by placing the auxiliary sheet between the two; suppresses re-moisturizing of the skin due to re-wetting; and reduces induction into a rough dry skin caused by re-contact with a body fluid such as menstrual blood.

Moreover, the labia are engaged in the through-hole, and this prevents the body fluid from diffusing and prevents the skin from becoming a rough dry skin in a wide range, and also reduces occurrence of the body fluid leakage from the sheet-form absorbent article.

1. An auxiliary sheet for an absorbent article which is to be used together with a sheet-form absorbent article, and supplements function of a sheet-form absorbent article or provides new function to the sheet-form absorbent article, said auxiliary sheet for the absorbent article is provided with a through-hole for smoothly engaging therein a predetermined range of a pair of labia, and
a depth of said through-hole is set deeper than that of a pair of said labia to be engaged in said through-hole when the through-hole of said auxiliary sheet for the absorbent article is engaged with a pair of said labia.

2. The auxiliary sheet for the absorbent article according to claim 1, wherein said auxiliary sheet for the absorbent article comprises at least one layer, said layer comprises at least one of a liquid impermeable sheet and a soft touch sheet.

3. The auxiliary sheet for the absorbent article according to claim 2, wherein the liquid impermeable sheet has air permeability.

4. The auxiliary sheet for the absorbent article according to claim 2, wherein said soft touch sheet comprises at least one of a nonwoven fabric sheet and a foam sheet.

5. The auxiliary sheet for the absorbent article according to claim 1, wherein said through-hole has an oblong elliptic opening.

6. The auxiliary sheet for the absorbent article according to claim 1, wherein said through-hole is biased toward either side of both end portions in the longitudinal direction of said auxiliary sheet for the absorbent article.

7. The auxiliary sheet for the absorbent article according to claim 1, having an adhesive portion for adhering to body of a wearer on a body side face of the auxiliary sheet for the absorbent.

8. The auxiliary sheet for the absorbent article according to claim 7, wherein said adhesive portion is formed in a shape of a belt passing in the vicinity of a circumferential portion of said through-hole, or said adhesive portion is formed along said circumferential portion thereof.

9. The auxiliary sheet for the absorbent article according to claim 7, further comprising an elastic sheet on a body side face of the auxiliary sheet for the absorbent article, and said adhesive portion is placed on the elastic sheet.

10. The auxiliary sheet for the absorbent article according to claim 9, wherein said auxiliary sheet for the absorbent article is provided with a plurality of long grooves having a recess shape in parallel in the longitudinal direction in a range where said elastic sheet is placed.

11. The auxiliary sheet for the absorbent article according to claim 1, further comprising a body fluid guiding stem for guiding a body fluid from said through-hole to said sheet-form absorbent article.

12. The auxiliary sheet for the absorbent article according to claim 11, wherein said body fluid guiding stem has a bottom formed on the opposite side to the body side.

13. The auxiliary sheet for the absorbent article according to claim 1, further comprising a belt-like fixing tab for fixing the auxiliary sheet on said sheet-form absorbent article at both sides of the longitudinal direction of said auxiliary sheet for the absorbent article are provided with.

14. The auxiliary sheet for the absorbent article according to claim 1, wherein said auxiliary sheet for the absorbent article is an auxiliary sheet for the absorbent article used for incontinence of urine.

15. The auxiliary sheet for the absorbent article according to claim 1, wherein said auxiliary sheet for the absorbent article is an auxiliary sheet for the absorbent article used for absorbing a vaginal discharge.
